(19) United States
(12) Patent Application Publication
(10) Pub. No.: US 2024/0180610 A1
(43) Pub. Date: Jun. 6, 2024

(54) SYSTEM AND METHOD FOR CARRYING OUT A MEDICAL PROCEDURE

(71) Applicant: Boston Scientific Medical Device Limited, Galway (IE)

(72) Inventor: Eduardo Moriyama, Richmond (CA)

(21) Appl. No.: 18/441,474

(22) Filed: Feb. 14, 2024

Related U.S. Application Data
(63) Continuation of application No. 16/794,799, filed on Feb. 19, 2020, now Pat. No. 11,931,098.

(51) Int. Cl. A61B 18/14; A61B 5/1455; A61B 5/1459; A61B 17/00; A61B 18/00; A61B 90/30; A61M 25/09

(52) U.S. Cl. CPC A61B 18/1477; A61B 5/14552; A61B 5/1459; A61B 2017/00057; A61B 2017/00119; A61B 2017/00247; A61B 2018/0038; A61B 2018/00601; A61B 2090/306; A61M 25/09

(57) ABSTRACT

A medical system includes a medical device and an analysis system. The medical device includes a perforation device having a shaft. The shaft has a proximal portion, and an opposed distal portion. A perforating tip is associated with the distal portion. At least a first light emitter is associated with the distal portion and is positioned proximate the perforating tip for illuminating a region surrounding the perforating tip. The analysis system is for analyzing returned light that is returned towards the shaft from the region surrounding the perforating tip. The analysis system includes a light sensor configured to detect one or more parameters of the returned light, a processor configured to perform an analysis of the one or more parameters, and an alert system connected to the processor for alerting a user to a result of the analysis.

### CROSS REFERENCE TO RELATED APPLICATIONS

The present application is a continuation of and claims benefit of U.S. patent application Ser. No. 16/794,799, entitled “SYSTEM AND METHOD FOR CARRYING OUT A MEDICAL PROCEDURE,” filed Feb. 19, 2020, the entirety of which is incorporated herein by reference.

### FIELD

This document relates to medical procedures such as transseptal perforation. More specifically, this document relates to devices for use in such medical procedures, and related systems and methods.

### SUMMARY

The following summary is intended to introduce the reader to various aspects of the detailed description, but not to define or delimit any invention.

Perforation devices for use in medical procedures are disclosed. According to some aspects, a perforation device includes a shaft. The shaft has a proximal portion, and an opposed distal portion. A perforating tip is associated with the distal portion. At least a first light emitter is associated with the distal portion and is positioned proximate the perforating tip, for illuminating a region surrounding the perforating tip. At least a first light collector is associated with the distal portion and is positioned proximate the perforating tip, for collecting light from the region surrounding the perforating tip.

In some examples, the perforation device further includes at least a first illumination optical fiber extending through the shaft from the proximal portion to the distal portion. The illumination optical fiber has a source end for receiving light from a light source and an illumination end proximate the perforating tip. The illumination end forms the light emitter.

In some examples the perforation device further includes at least a first collection optical fiber extending through the shaft from the proximal portion to the distal portion. The collection optical fiber has a light collection end that forms the light collector, and a sensor end for delivering light to a light sensor.

In some examples, the light collector is in the form of a light sensor associated with the distal portion of the shaft.

In some examples, the perforating tip includes a radiofrequency perforation electrode.

In some examples, the illumination end is shrouded within the shaft, and the perforation device further includes a light scattering material between the illumination end and an opening in the shaft for directing light from the illumination end to an exterior of the shaft.

In some examples, the illumination end is distally facing. In some examples, the illumination end is recessed proximally from a distal end face of the shaft. In some examples, the illumination end is flush with a distal end face of the shaft.

Medical systems are also disclosed. According to some aspects, a medical system includes a perforation device. The perforation device includes a perforation device having a shaft. The shaft has a proximal portion, and an opposed distal portion. A perforating tip is associated with the distal portion. At least a first light emitter is associated with the distal portion and is positioned proximate the perforating tip for illuminating a region surrounding the perforating tip. The medical system further includes an analysis system for analyzing returned light that is returned back towards the shaft from the region surrounding the perforating tip. The analysis system includes a light sensor configured to detect one or more parameters of the returned light, a processor configured to perform an analysis of the one or more parameters, and an alert system connected to the processor for alerting a user to a result of the analysis.

In some examples, the medical system includes a light source, and at least a first illumination optical fiber extending through the shaft from the proximal portion to the distal portion. The illumination optical fiber has a source end connected to the light source for receiving light from the light source, and an illumination end positioned proximate the perforating tip and forming the light emitter.

In some examples, the medical system further includes a first collection optical fiber extending through the shaft from the proximal portion to the distal portion. The collection optical fiber has a light collection end for collecting the returned light, and a sensor end for delivering light to the sensor.

In some examples, the sensor is mounted to the distal portion of the shaft.

In some examples, the result is an indication of whether blood in the region surrounding the perforating tip is oxygenated blood or deoxygenated blood.

In some examples, the perforating tip includes a radiofrequency perforation electrode. The medical system can further include a radio frequency generator connected to the radiofrequency perforation electrode for powering the radiofrequency perforation electrode. The generator can be in communication with the analysis system and can be configured to provide power to the radiofrequency perforation electrode based on the analysis. The generator can be configured to cease providing power to the radiofrequency perforation electrode if the analysis indicates that the region surrounding the perforating tip contains oxygenated blood.

Methods for carrying out medical procedures are also disclosed. According to some aspects, a method for carrying out a medical procedure includes a. positioning a perforating tip of a puncture device adjacent a target region within a patient's body; b. advancing the perforating tip through the target region; c. before, during and/or after step b., illuminating a region surrounding the perforating tip with light, collecting light returned back from the region surrounding the perforating tip, and analyzing the returned light.

In some examples, the method further includes ceasing advancement of the perforating tip if the analysis of the returned light indicates that the region surrounding the perforating tip contains oxygenated blood.

In some examples, the method further includes continuing or repeating advancement of the perforating tip if the analysis of the returned light indicates that the region surrounding the perforating tip contains deoxygenated blood.

In some examples, step b. includes delivering radiofrequency energy to the perforating tip to puncture the fossa ovalis. In some examples, the method further includes ceasing the delivery of radiofrequency energy if the analysis of the returned light indicates that the region surrounding the perforating tip contains oxygenated blood. In some examples, the target region is a fossa ovalis.

### BRIEF DESCRIPTION OF THE DRAWINGS

The accompanying drawings are for illustrating examples of articles, methods, and apparatuses of the present disclosure and are not intended to be limiting. In the drawings:

FIG. 1 shows an example absorption spectrum for oxyhemoglobin and hemoglobin;

FIG. 2 is a perspective view of an example medical system;

FIG. 3 is a perspective view of the perforation device of the medical system of FIG. 2;

FIG. 4 is a partial side view of the perforation device of FIG. 3;

FIG. 5 is an end view of the perforation device of FIG. 3;

FIG. 6 is a partial side view of another example perforation device;

FIG. 7 is an end view of the perforation device of FIG. 6;

FIG. 8 is a is a partial side view of another example perforation device;

FIG. 9 is an end view of the perforation device of FIG. 8;

FIG. 10 is a longitudinal cross-section taken through another example perforation device;

FIG. 11 is transverse cross-section taken through the perforation device of FIG. 10;

FIG. 12 is a longitudinal cross-section taken through another example perforation device;

FIG. 13 is transverse cross-section taken through the perforation device of FIG. 12;

FIG. 14 shows a step of a method using the system of FIG. 2;

FIG. 15 shows another step of a method using the system of FIG. 2; and

FIG. 16 shows another step of a method using the system of FIG. 2.

### DETAILED DESCRIPTION

Various apparatuses or processes or compositions will be described below to provide an example of an embodiment of the claimed subject matter. No example described below limits any claim and any claim may cover processes or apparatuses or compositions that differ from those described below. The claims are not limited to apparatuses or processes or compositions having all of the features of any one apparatus or process or composition described below or to features common to multiple or all of the apparatuses or processes or compositions described below. It is possible that an apparatus or process or composition described below is not an embodiment of any exclusive right granted by issuance of this patent application. Any subject matter described below and for which an exclusive right is not granted by issuance of this patent application may be the subject matter of another protective instrument, for example, a continuing patent application, and the applicants, inventors or owners do not intend to abandon, disclaim or dedicate to the public any such subject matter by its disclosure in this document.

Generally disclosed herein are devices and systems that can be used in medical procedures, such as cardiac procedures. For example, the devices systems can be used in transseptal perforation procedures, in which a sheath is advanced to the right atrium of a patient's heart via the femoral vein, and a perforation device (e.g. a radiofrequency (RF) perforation device or a mechanical perforation device) and dilator are guided through the sheath, to the right atrium. When the sheath is adjacent a target region in the right atrium, for example the fossa ovalis of the atrial septum, the perforation device can be advanced out of the sheath and used to create a perforation in the target region, and the dilator can be advanced out of the sheath to dilate the perforation. Such procedures can be carried out, for example, as a medical treatment, or to gain access to the left atrium for a subsequent medical treatment.

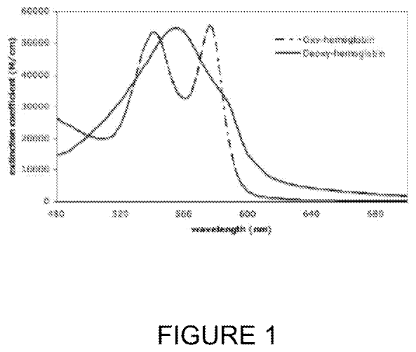

The systems, devices, and methods disclosed herein can be used to determine whether perforation is complete—i.e. whether the perforation device has indeed perforated the atrial septum and the perforating tip is in the left atrium, or whether perforation is not yet complete and the perforating tip is within the atrial septum, still within the right atrium, or elsewhere. This can in turn reduce the use of fluoroscopy, decrease the complication rate in such procedures, and enhance patient safety. Particularly, the systems and devices disclosed herein are configured to emit light to (i.e. illuminate) the region surrounding the perforating tip of the perforation device, and to collect light that is returned from the region surrounding the perforating tip (referred to herein as “returned light”, which can be, for example, reflected light or emitted fluorescence). This returned light is then analyzed, for example using absorption or fluorescence spectroscopy. Particularly, blood that is within the right atrium is venous blood, which is not oxygenated and largely contains deoxyhemoglobin. In contrast, blood that is within the left atrium is arterial blood, which is oxygenated and largely contains oxyhemoglobin. Deoxyhemoglobin and oxyhemoglobin have distinct absorption spectra in the range of 520 nm and 600 nm, as shown in FIG. 1. By analyzing the absorption spectrum of the light returned from the region surrounding the perforating tip, it can be determined whether this region largely contains deoxyhemoglobin or oxyhemoglobin, and in turn it can be determined whether the perforating tip is in the right atrium (which contains largely deoxyhemoglobin) or the left atrium (which contains largely oxyhemoglobin).

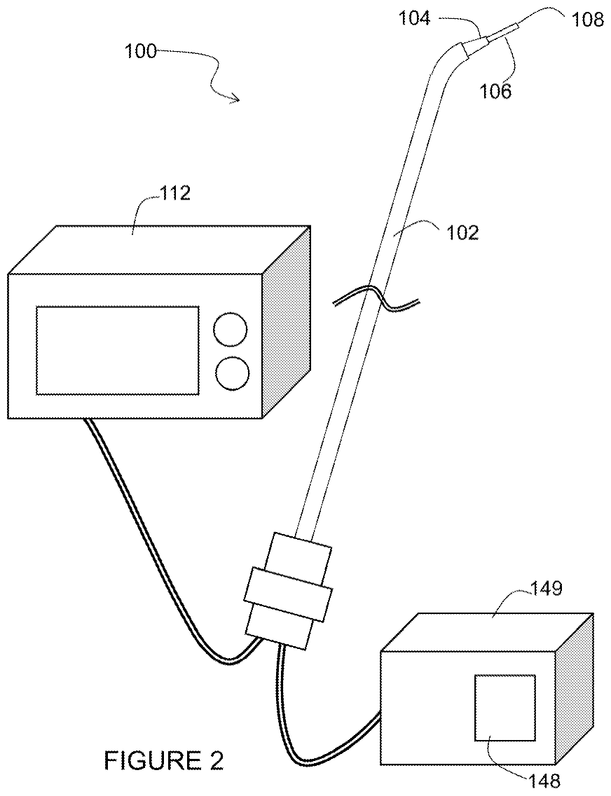

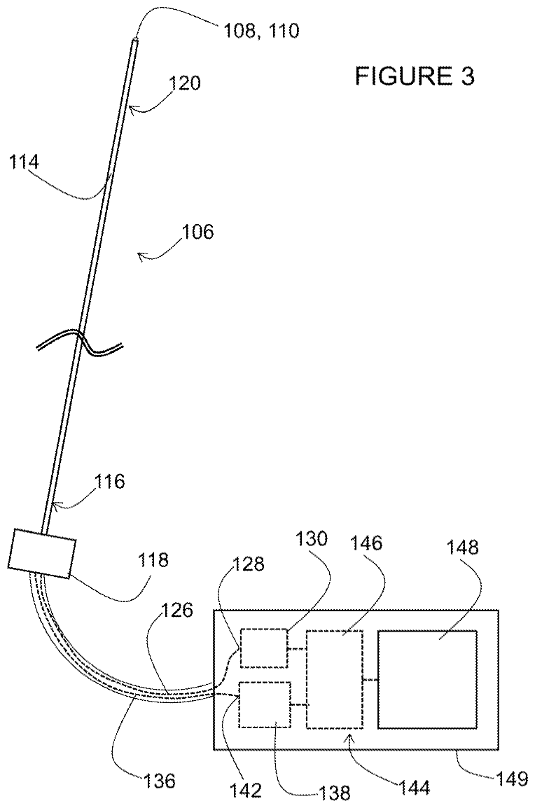

Referring now to FIG. 2, an example medical system 100 is shown. In the example shown, the system 100 is a transseptal perforation system, for advancing towards a patient's heart and perforating a fossa ovalis of the patient's heart. The system includes a set of medical devices, including a sheath 102, a dilator 104, and a perforation device 106 having a perforating tip 108. In the examples shown, the perforation device 106 is a radiofrequency (RF) perforation device, and the perforating tip 108 includes a radiofrequency electrode 110 (shown in FIG. 3).

In use, the sheath 102 can be advanced intravenously via the femoral vein towards the right atrium of the patient's heart. The dilator 104 and perforation device 106 can both be advanced towards the patient's heart via the sheath 102. The RF perforation device 106 can be connected to a radiofrequency generator 112, which can in turn be connected to one or more grounding pads (not shown). When in the desired position in the patient's heart, for example adjacent the fossa ovalis, the RF perforation device 106 can be activated to perforate the fossa ovalis.

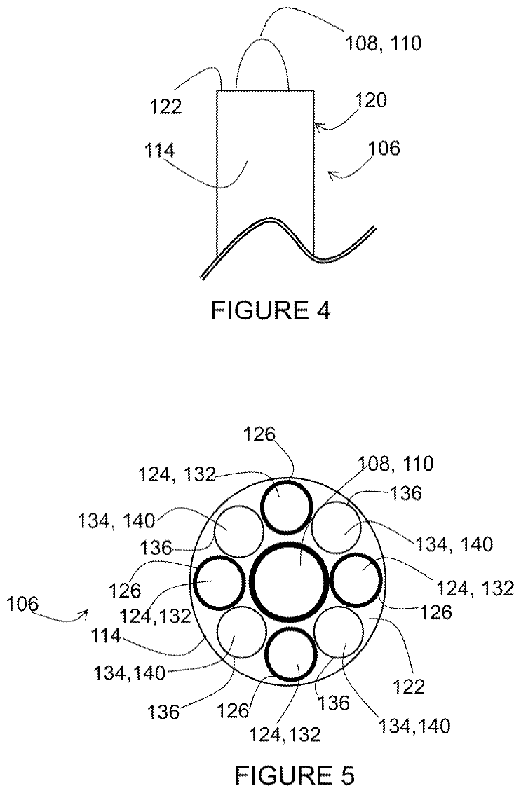

Referring to FIGS. 3 to 5, the perforation device 106 is shown in greater detail. In the example shown, the perforation device 106 includes a shaft 114, which can be, for example, fabricated from a plastic. The shaft 114 has a proximal portion 116 that is connected to a hub 118, and an opposed distal portion 120. The perforating tip 108 is associated with the distal portion 120 of the shaft 114. As used herein, the term “associated with” means that the first referenced part (i.e. in this case the perforating tip 108) and second referenced part (i.e. in this case the shaft 114) are configured so that the first referenced part moves with the second referenced part. For example, the first referenced part can be mounted to, extend from, adhered to, embedded in, part of, formed by, and/or integral with second referenced part. In the example shown, the perforating tip 108 includes the radiofrequency perforation electrode 110, which is secured to and extends distally from a distal face 122 of the shaft 114. An electrical connector (not shown) is connected to and extends proximally from the radiofrequency perforation electrode 110 through the shaft 114, for connection to the radiofrequency generator 112 (not shown in FIGS. 3 to 5)

The perforation device 106 further includes a set of light emitters 124 associated with the distal portion 120 of the shaft 114 and positioned proximate the perforating tip 108 for illuminating the region surrounding the perforating tip 108 (i.e. for delivering light to blood or tissue that is in contact with or near to the perforating tip 108). The light emitters 124 can be of various configurations. Referring to FIG. 5, in the example shown, the perforation device 106 includes a first set of optical fibers, referred to herein to as “illumination optical fibers 126”. The illumination optical fibers 126 are embedded in the shaft 114 and extend through the shaft 114 from the proximal portion 116 to the distal portion 120. For example, the illumination optical fibers 126 can be embedded in dedicated channels within the shaft 114. In alternative examples, the shaft 114 can have a lumen, and the illumination optical fibers 126 can extend through the lumen, and be sealed within the lumen by an epoxy or glue.

Referring to FIG. 3 (in which only one of the illumination optical fibers 126 is shown, and is shown only partially), the illumination optical fibers 126 each have a first end 128 (referred to herein as a “source end) that is connected or connectable to a light source 130 (described below), for receiving light from the light source 130. Referring back to FIG. 5, the illumination optical fibers 126 further have a second end 132 (referred to herein as an “illumination end”) proximate the perforating tip 108. The illumination end 132 of each illumination optical fiber 126 forms a light emitter 124, and illuminates the region surrounding the perforating tip 108. In the example shown, the illumination end 132 of each illumination optical fiber 126 is flush with the distal end face 122 of the shaft 114, and is distally facing. In alternative examples, the illumination end 132 of the illumination optical fibers 126 can be recessed proximally from the distal end face 122 of the shaft 114, or can extend proud of the distal end face 122 of the shaft 114.

Referring still to FIG. 5, the perforation device 106 further includes a set of light collectors 134 associated with the distal portion 120 of the shaft 114 and positioned proximate the perforating tip 108, for collecting light returned from the region surrounding the perforating tip 108 (also referred to herein as “returned light”). For example, the returned light can be reflected light or emitted fluorescence. The light collectors 134 can be of various configurations. In the example shown, the perforation device 106 includes a second set of optical fibers, referred to herein as “collection optical fibers 136”, for collecting light returned from the region surrounding the perforating tip 108 and delivering the returned light to a light sensor 138 (described below). The collection optical fibers 136 are embedded in the shaft 114 and extend through the shaft 114 from the distal portion 120 to the proximal portion 116. For example, the collection optical fibers 136 can be embedded in dedicated channels within the shaft 114. In alternative examples, the shaft 114 can have a lumen, and the collection optical fibers 136 can extend through the lumen, and be sealed within the lumen by an epoxy or glue.

Referring still to FIG. 5, the collection optical fibers 136 each have a first end 140 (referred to herein as a “light collection end”) that forms the light collector 134. In the example shown, the collection end 140 of the collection optical fibers 136 is flush with the distal end face 122 of the shaft 114, and is distally facing. In alternative examples, the collection end 140 of the collection optical fibers 136 can be recessed proximally from the distal end face 122 of the shaft 114, or can extend proud of the distal end face 122 of the shaft 114. Referring to FIG. 3 (in which only one of the collection optical fibers 136 is shown, and is shown only partially), the collection optical fibers 136 further have a second end 142 (referred to herein as an “sensor end”) for delivering light to the light sensor 138.

Referring still to FIGS. 3 to 5, in the example shown, the illumination optical fibers 126 and collection optical fibers 136 are arranged concentrically around the RF electrode 110 in an alternating pattern. In other examples, as will be described below, the illumination optical fibers 126 and collection optical fibers 136 can be of another arrangement.

Referring still to FIG. 3, the light source 130 can be, for example, a laser, a lamp (e.g. an Xe or Arc lamp), or an LED. The light source 130 can be monochromatic or broad band. The light delivered by the light source 130 can be continuous or pulsed (e.g. pulsed by a pulse generator or light chopper).

Referring still to FIG. 3, the system 100 includes an analysis system 144 for analyzing the returned light. The analysis system 144 includes the light sensor 138, which can be any suitable sensor that can detect one or more parameters (e.g. the absorption or fluorescence spectrum) of the returned light. For example, the light sensor 138 can be or can include a photodiode, a power meter, a spectrometer, a phototransistor, and/or a photomultiplier. The light sensor 138 can be a single chip or multi-chip detector. The light sensor 138 can include a CCD or CMOS camera.

The analysis system 144 further includes a processor 146 connected to the light sensor. The processor 146 can include various hardware and software, and is configured to perform an analysis of the parameters detected by the light sensor 138. For example, the processor 146 can compare the absorption spectrum of the returned light to a stored reference absorption spectrum for oxyhemoglobin and deoxyhemoglobin. Based on this comparison, the processor 146 can then determine whether the absorption spectrum of the returned light corresponds to the reference spectrum for oxyhemoglobin (which indicates that the region surrounding the perforating tip 108 contains oxygenated blood) or deoxyhemoglobin (which indicates that the region surrounding the perforating tip 108 contains deoxygenated blood).

The analysis system 144 further includes an alerting system connected to the processor 146, for alerting a user to a result of the analysis. In the example shown, the alerting system includes a screen 148 that displays an indicium of the position of the perforating tip 108. Alternatively, the alerting system can be, for example, in the form of a light that changes color when the analysis indicates that the region surrounding the perforating tip 108 contains oxygenated blood. The change in color of the light can indicate to a user that the perforating tip 108 is in the left atrium. Alternatively, the alerting system can provide an auditory alert that indicates to a user that the perforating tip 108 is in the left atrium.

In the example shown, the light source 130, light sensor 138, processor 146, and screen 148 are provided in a single unit 149. In alternative examples, the light source 130, light sensor 138, processor 146, and screen 148 may be provided in separate units or at separate locations. For example, the light sensor 138 may be mounted to the perforation device 106 (as described below), and the processor 146 and screen 148 may be provided in a separate unit. Alternatively, one or more of the light source 130, light sensor 138, processor 146, and screen 148 may be provided in a unit with the RF generator 112.

Optionally, the RF generator 112 (shown in FIG. 2) can be in communication with the analysis system 144, and can be configured to provide power to the RF electrode 110 based on the analysis. For example, the RF generator 112 can be configured to provide power to the RF electrode 110 only while the analysis indicates that the region surrounding the perforating tip 108 contains deoxygenated blood. Alternatively, the RF generator 112 can be configured to cease providing power to the RF electrode 110 if the analysis indicates that the region surrounding the perforating tip 108 contains oxygenated blood. This may further enhance safety, as once perforation of the fossa ovalis is complete, perforation of additional structures will be prevented (or the risk thereof will be reduced).

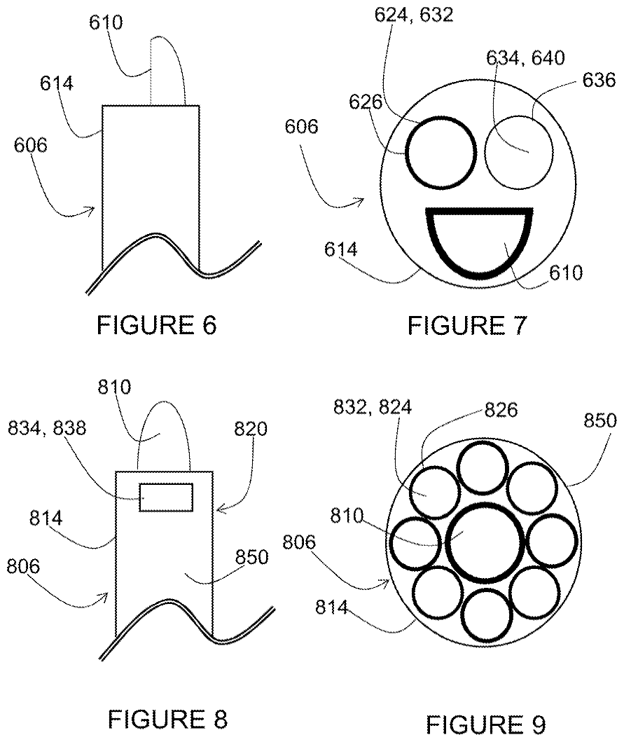

Referring now to FIGS. 6 and 7, another example of a perforation device is shown. In FIGS. 6 and 7, features that are similar to those of FIGS. 1 to 5 are referred to with like reference numerals, incremented by 500. The perforation device 606 of FIGS. 6 and 7 includes only one light emitter 624, in the form of an illumination end 632 of an illumination optical fiber 626, and only one light collector 634, in the form of a collection end 640 of a collection optical fiber 636. The light emitter 624 and light collector 634 are positioned side-by-side and adjacent the RF electrode 610, and are and off-centre within the shaft 614. The illumination optical fiber 626 and collection optical fiber 636 have a larger diameter than the corresponding parts of FIGS. 1 to 5.

Referring now to FIGS. 8 and 9, another example of a perforation device is shown. In FIGS. 8 and 9, features that are similar to those of FIGS. 1 to 5 are referred to with like reference numerals, incremented by 700. In the example of FIGS. 8 and 9, similarly to the example of FIGS. 1 to 5, the perforation device 806 includes a set of illumination optical fibers 826, the illumination ends 832 of which form light emitters 824 (only one of the illumination optical fibers 826 is labelled in FIG. 9). The illumination optical fibers 826 are arranged concentrically around the RF electrode 810.

In the example of FIGS. 8 and 9, collection optical fibers are omitted, and the light collector 834 is in the form of a light sensor 838 associated with the distal portion 820 of the shaft 814. In the example shown, the light sensor 838 is mounted to a side surface 850 of the shaft 814. An electrical connector (not shown) extends through the shaft 814 to connect the light sensor 838 to the processor (not shown) of the analysis system (not shown). The light sensor 838 can be any suitable sensor that can detect one or more parameters (e.g. the absorption or fluorescence spectrum) of the returned light. For example, the light sensor 838 can be or can include a photodiode, a power meter, a spectrometer, a phototransistor, and/or a photomultiplier. The light sensor 838 can be a single chip or multi-chip detector. The light sensor 838 can include a CCD or CMOS camera.

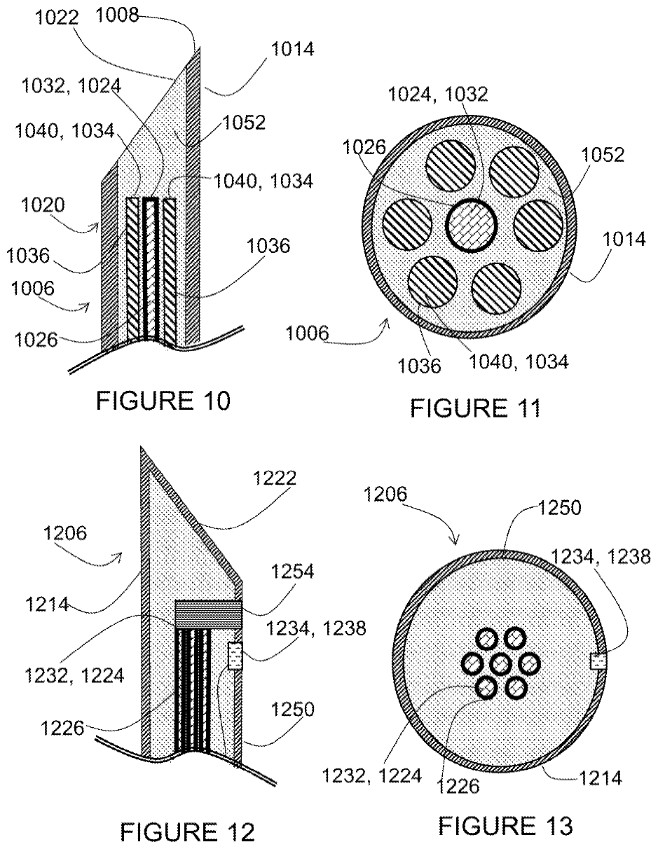

Referring now to FIGS. 10 and 11, another example of a perforation device is shown. In FIGS. 10 and 11, features that are similar to those of FIGS. 1 to 5 are referred to with like reference numerals, incremented by 900. In the example of FIGS. 10 and 11, the perforation device 1006 is a mechanical perforation device, in which the distal portion 1020 of the shaft 1014 is beveled to provide a sharp perforating tip 1008. The perforation device 1006 includes an illumination optical fiber 1026, the illumination end 1032 of which forms a light emitter 1024, and a set of collection optical fibers 1036 (only one of which is labelled in FIG. 11), the collection ends 1040 of which form light collectors 1034. The illumination optical fiber 1026 and the collection optical fibers 1036 are positioned within a lumen of the shaft 1014, and the illumination end 1032 and collection ends 1040 are recessed proximally from the distal end face 1022 of the shaft 1014. A light transmitting material 1052 such as an epoxy or glue seals the lumen.

In the example of FIGS. 10 and 11, the perforating device 1006 includes a single illumination optical fiber 1026 and six collection optical fibers 1036. In other examples, another number of illumination optical fibers and collection fibers can be used.

Referring now to FIGS. 12 and 13, another example of a perforation device is shown. In FIGS. 12 and 13, features that are similar to those of FIGS. 1 to 5 are referred to with like reference numerals, incremented by 1100. In the example of FIGS. 12 and 13, the perforation device 1206 includes a set of illumination optical fiber 1226 (only one of which is labelled), the illumination ends 1232 of which form light emitter 1224. The illumination ends 1232 are shrouded within the shaft 1214, the distal end face 1222 of which is closed. A light scattering material 1254 is positioned within the shaft 1214, between the illumination ends 1232 and an opening on the side surface 1250 of the shaft 1214. The light scattering material 1254 directs light radially from the illumination ends 1232 to the exterior of the shaft 1214.

In the example of FIGS. 12 and 13, the light collector 1234 is in the form of a light sensor 1238 that is mounted to the side surface 1250 of the shaft 1214, proximate to the light scattering material 1254.

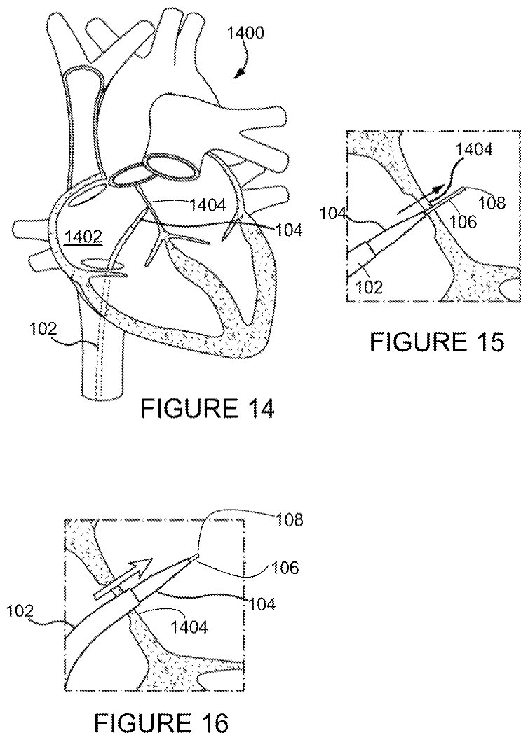

A method for transseptal perforation will now be described with reference to FIGS. 14 to 16. The method will be described with reference to the system of FIGS. 1 to 5; however, the system of FIGS. 1 to 5 can be used according to different methods, and the method can employ different systems.

As a first step (not shown), a guidewire can be advanced via the femoral vein towards the heart, and “parked” in the superior vena cava (SVC). The dilator 104 can then be inserted into the sheath 102, with the tip of the dilator 104 shrouded within the sheath 102. The sheath 102 and dilator 104 can then be advanced over the guidewire, towards the SVC. The guidewire can then be removed.

As a second step (not shown), the perforation device 106 can be advanced through the dilator 104, until the perforating tip 108 is just shy of the distal end of the dilator 104.

Referring to FIG. 14, as a third step, the distal end of the sheath 102 can then be advanced towards a target location in the patient's heart 1400, e.g. to the right atrium 1402 of the patient's heart 1400, to position the distal end of the sheath 102 adjacent the target location. In the example shown, the target location is the fossa ovalis 1404 of the atrial septum. The dilator 104 can then be advanced so that the dilating end thereof is proud of the sheath 102, and the perforation device 106 (not visible in FIG. 14) can be advanced so that the perforating tip 108 is proud of or flush with the dilator 104 and is adjacent the fossa ovalis 1404. This positioning is shown in FIG. 14

With the perforation device 106 in the ready position, as shown in FIG. 14, The light source 130 (not shown in FIGS. 14 to 16) and analysis system 144 (not shown in FIGS. 14 to 16) can then be engaged, to confirm the positioning of the perforating tip 108. Particularly, the light source 130 can be engaged to illuminate the region surrounding the perforating tip 108, and the returned light can be collected and analyzed to determine whether the region surrounding the perforating tip 108 contains oxygenated blood or deoxygenated blood. If the analysis indicates that the region surrounding the perforating tip 108 contains oxygenated blood, this indicates that the perforating tip 108 is not properly positioned, and the procedure can be stopped or the position of the perforation device 106 can be adjusted or checked using imaging. If the analysis indicates that the region surrounding the perforating tip 108 contains deoxygenated blood, this indicates that the perforating tip 108 is properly positioned, and the procedure can continue.

To continue the procedure, the RF generator 112 (not shown in FIGS. 14 to 16) can be engaged, to supply power to the RF electrode 110. The perforating tip 108 can then be advanced through the fossa ovalis 1404, as shown in FIG. 15. During and/or after advancement of the perforating tip 108, the position of the perforating tip 108 can be monitored and/or confirmed. That is, the light source 130 can be engaged to illuminate the region surrounding the perforating tip 108, and the returned light can be collected and analyzed to determine whether the region surrounding the perforating tip 108 contains oxygenated blood or deoxygenated blood. If the analysis indicates that the region surrounding the perforating tip 108 contains oxygenated blood, this indicates that the perforating tip 108 has perforated the fossa ovalis 1404 and is in the left atrium, and the advancement of the perforating tip 108 and the delivery of RF energy can be ceased. If the analysis indicates that the region surrounding the perforating tip 108 contains deoxygenated blood, this indicates that the perforating tip 108 has not perforated the fossa ovalis 1404, and advancement of the perforating tip 108 and delivery of RF energy should continue or be repeated.

Optionally, before or during advancement of the perforating tip 108 through the fossa ovalis, the fluorescence spectra of the tissue of the fossa ovalis 1404 can be obtained using an excitation wavelength of about 365 nm. Due to the presence of collagen and other fluorophores within the fossa ovalis 1404, a distinct fluorescence spectrum may be obtained at this wavelength. This can be used to confirm that the perforating tip is within or in contact with the fossa ovalis 1404.

Referring to FIG. 16, once the fossa ovalis 1404 has been perforated, the dilator 104 can be advanced from the sheath 102 to dilate the perforation, and the sheath 102 can then be advanced through the perforation, to the left atrium. Once access to the left atrium has been gained, a subsequent medical treatment (not shown) can be carried out.

In alternative examples, other anatomical regions can be perforated using the devices, systems, and methods disclosed herein. For example, the devices, systems, and methods disclosed herein can be used to create channels between vessels (i.e. artery to vein or vice versa) and/or in ventricular puncture.

While the above description provides examples of one or more processes or apparatuses or compositions, it will be appreciated that other processes or apparatuses or compositions may be within the scope of the accompanying claims.

To the extent any amendments, characterizations, or other assertions previously made (in this or in any related patent applications or patents, including any parent, sibling, or child) with respect to any art, prior or otherwise, could be construed as a disclaimer of any subject matter supported by the present disclosure of this application, Applicant hereby rescinds and retracts such disclaimer. Applicant also respectfully submits that any prior art previously considered in any related patent applications or patents, including any parent, sibling, or child, may need to be re-visited.

We claim:

1. A perforation device for use in transseptal perforation procedures, comprising:
a shaft having a proximal portion and an opposed distal portion, the distal portion including a distal face;
a radio frequency perforating tip coupled to the distal portion and extending distally from the distal face;
an illumination end for illuminating a region surrounding the perforating tip, the illumination end disposed on the distal face; and
at least a first light collector associated with the distal portion and positioned proximate the perforating tip for collecting light from the region surrounding the perforating tip.

2. The perforation device of claim 1, further comprising at least a first illumination optical fiber extending through the shaft from the proximal portion to the distal portion, the illumination optical fiber having a source end for receiving light from a light source, wherein the illumination end forms the light emitter.

3. The perforation device of claim 1, further comprising at least a first collection optical fiber extending through the shaft from the proximal portion to the distal portion, the collection optical fiber having a light collection end that forms the light collector, and a sensor end for delivering light to a light sensor.

4. The perforation device of claim 1, wherein the light collector is in the form of a light sensor associated with the distal portion of the shaft.

5. The perforation device of claim 1, wherein the illumination end is shrouded within the shaft, and the perforation device further comprises a light scattering material between the illumination end and an opening in the shaft for directing light from the illumination end to an exterior of the shaft.

6. The perforation device of claim 1, wherein the illumination end is distally facing.

7. The perforation device of claim 1, wherein the illumination end is recessed proximally from a distal end face of the shaft.

8. The perforation device of claim 1, wherein the illumination end is flush with a distal end face of the shaft.

9. The perforation device of claim 1, wherein the shaft includes a longitudinal lumen and further comprising a dilator adapted to be advanced through the longitudinal lumen.

10. A medical system for providing transseptal punctures, the medical system comprising:
a perforation device including a shaft having a longitudinal axis and extending from a proximal portion to a distal portion, a perforating tip coupled to the distal portion, and a first light emitter associated with the distal face of the distal portion and positioned proximate the perforating tip for illuminating a region surrounding the perforating tip;
wherein the distal portion includes a distal face perpendicular to the longitudinal axis; and
an analysis system for analyzing light returned from the region surrounding the perforating tip, the analysis system including a light sensor configured to detect one or more parameters of the returned light, a processor configured to perform an analysis of the one or more parameters, and an alert system for alerting a user to a result of the analysis.

11. The medical system of claim 10, further comprising a light source, and at least a first illumination optical fiber extending through the shaft from the proximal portion to the distal portion, the illumination optical fiber having a source end connected to the light source for receiving light from the light source, and an illumination end positioned proximate the perforating tip and forming the light emitter.

12. The medical system of claim 10 wherein the perforation device further comprises a first collection optical fiber extending through the shaft from the proximal portion to the distal portion, the collection optical fiber having a light collection end for collecting the returned light, and a sensor end for delivering light to the sensor.

13. The medical system of claim 10, wherein the sensor is mounted to the distal portion of the shaft.

14. The medical system of claim 10, wherein one of the one or more parameters of analysis of the returned light is whether blood in the region surrounding the perforating tip is oxygenated blood or deoxygenated blood.

15. The medical system of claim 10, wherein the medical system further comprises a radio frequency generator connected to the radiofrequency perforation electrode for powering the radiofrequency perforation electrode; and the generator is in communication with the analysis system and is configured to provide power to the radiofrequency perforation electrode based on the analysis.

16. The medical system of claim 15, wherein the generator is configured to cease providing power to the radiofrequency perforation electrode if the analysis indicates that the region surrounding the perforating tip contains oxygenated blood.
